(19) United States
(12) Patent Application Publication
(10) Pub. No.: US 2016/0331513 A1
(43) Pub. Date: Nov. 17, 2016

(54) MULTI TEXTURED PROSTHESIS IMPLANT SHELL

(71) Applicant: Michael J. Brown, Ashburn, VA (US)

(72) Inventor: Michael J. Brown, Ashburn, VA (US)

(21) Appl. No.: 14/713,912

(22) Filed: May 15, 2015

(51) Int. Cl. A61F 2/12

(52) U.S. Cl. CPC A61F 2/12; A61F 2210/0076; A61F 2230/0071

(57) ABSTRACT

A breast prosthesis, including an elastomeric shell including an elastomeric shell including a front exterior surfaces including a first portion having a first surface texture, and a second portion having a second surface texture different than the first surface texture, and a back exterior surface having a third surface texture, and a front transition area that transitions between the first portion and the second portion of the front exterior surface.

### BACKGROUND OF THE INVENTION

1. Field of the Invention

The present invention relates to prosthetics, and more particularly to shaped breast implant prosthetics or expanders and their exterior surface compositions being both smooth and textured on the anterior surface and textured on the posterior surface.

2. Description of the Related Art

An internal breast implant prosthetic may be placed in the human body for breast augmentation and/or breast reconstruction. The indications for breast reconstruction include restoring breast volumes after tumor or cancer removal, as well as congenital breast developmental anomalies. An implant is typically constructed with a silicone elastomeric polymer shell. The shell is generally described as having two surfaces, a front side and a back side, also known as the anterior and posterior surfaces, respectively.

The shell is typically filled with either a physiologic solution or an inert silicone gel polymer. The solution-filled implants can be filled at a production factory or more commonly at the time of surgery. In contrast, the silicone gel filled devices are typically always filled at the production factory. Other types of fills have been invented but are not in mass production or use.

The primary differences in the related devices is the type and degree of rough texturing created during the production of the devices, the shell thickness, the shape, the dimensions, the existence of baffles, the various fills used for the devices, valve requirements, and fabrication specifications.

The overwhelming majority of related devices have the same surface texture on both sides of the device. Thus, the implants are either completely smooth textured devices or completely rough textured devices on both sides. For discussion purposes, most manufacturers and physicians will describe implants as either ‘smooth’ or ‘textured’, where the “textured” label is reserved for the more roughly textured devices.

The rough texturing has been shown to promote tissue ingrowth and adherence, as well as alter the scar tissue that forms around the implant. The scar tissue that forms around the implant is known as the “implant capsule.” This capsule is typically very thin and causes no adverse consequences. It is known to be a natural response to the prosthetic being placed into the body. When the capsule thickens, it can cause distortion of the breast mound and even cause pain. This adverse consequence is called a “capsular contracture.”

In an effort to reduce and prevent capsular contracture, related inventions introduced the textured shell to alter the scar tissue characteristics and make it more difficult for the capsule to progressively thicken and tighten around the implant. Other related inventions have focused on exterior shell compositions in efforts to reduce the capsular thickening and maintain the natural feeling and appearing breast mound. These other invention implant compositions have included dissolvable exterior shells components, different architecture of the texturing, fabrics applied to the shells, and shells that actually have chemical mediators placed into the shell to inhibit the scar tissue excess growth.

In sum, the textured shell has been shown to minimize the degree of severe scar tissue formation that surrounds the exterior surface of the prosthetic. The texturing with its tissue ingrowth also significantly contributes to tissue adherence to the implant and thus inhibits implant migration or undesirable repositioning the implant.

With these potential benefits, prior implants have covered both sides of the implant with the rough texturing. In prior implants, the manufacturing techniques used to create the texturing and the degree of texturing have been one of the primary focus concentrations. There has been very limited focus on whether the texturing is needed on both sides of the implant.

In regards to the texturing of prior implants, the majority have presented either completely smooth textured or roughly textured implant shells. There exist only a few prior implants which utilized at least two distinct textural surfaces. One prior technique depicts a roughly textured posterior surface and a smooth textured anterior surface. Another technique depicts a roughly textured posterior surface, with a smooth surface on the anterior surface and additional texturing described as a fixation surface for pectoralis major fixation.

The implants are also typically described or classified by their overall shape in one of two ways; either as a round device, or as an anatomic-shaped (or also known as tear-dropped shaped device). For purposes of this present invention, the discussion will reference the anatomic-shaped devices with similar applications found for round devices.

The prior anatomic shaped devices have had a history of rotating into an undesirable position which contributes to a misshaped breast mound over time. This misshaped contour occurs because the anatomic shaped devices have a specific orientation or longitudinal axis and when the implant is off its proper axis, the breast will take an unnatural contour. These anatomic devices may be placed above or below the pectoralis major muscle. The pectoralis major muscle contributes to camouflaging the implant and contributes to a more natural appearing breast mound. This ‘below the muscle’ position is typically the preferred implant placement position. This benefit also brings in a degree of complexity because with flexion of the pectoralis major muscle there is a stress placed on the implant. Since the pectoralis major muscle is a primary muscle for upper extremity (arm) use and range of motion, it is a very strong muscle. Pectoralis major muscle contraction originates from the sternum of the rib cage and along the ribs and ends on the upper arm near the shoulder. This contraction pulls the muscle tight across the upper chest and ultimately moves the upper arm. When an implant is placed beneath or partially beneath the pectoralis major muscle, it is exposed to the muscle contraction repeatedly throughout the day. It is this muscular contraction that can influence the implants position on the chest and hence the breast. This stress can contribute to implant migration by being squeezed out from under the muscle or rotating off of the implant's proper longitudinal axis. In an effort to prevent this migration or malrotation, texturing typically has been added to anatomic shaped devices.

It is believed that the anterior surface shell texturing on conventional structures that is under the muscle actually does not promote adherence to the muscle but actually acts detrimentally. With a muscular contraction, the implant may be incrementally displaced. Having the anterior surface texturing on the implant that comes in contact with undersurface of the pectoralis major muscle does not allow the implant to return to the more optimal axis or position because the muscle essentially has moved over the implant and grabs hold of the roughened surface and holds it in a slightly deviated position. Over time, these incremental movements result in visible contour changes. This can also occur with round-shaped devices with the implant being displaced.

### SUMMARY OF THE INVENTION

In view of the foregoing and other exemplary drawbacks of the conventional structures and techniques, the present invention aims to provide an implantable prosthesis for implantation into a human being. The implant is preferably for the human breast and may be used for augmentation of the breast; or reconstruction of the breast after congenital or acquired deformities of the breast.

Accordingly, it is an exemplary feature of the invention to provide an implantable prosthesis with multiple textures.

According to one exemplary embodiment of the invention, a breast prosthesis includes: an elastomeric shell including:
a front exterior surface including:
   a first portion having a first surface texture, and
   a second portion having a second surface texture different than the first surface texture,
a back exterior surface having a third surface texture; and
a front transition area that transitions between the first portion and the second portion of the front exterior surface.

The implantable prosthesis can be composed of a silicone elastomeric shell that is filled with an inert silicone gel or a physiologic solution. The implant has two primary exterior sides, the front and back of the implant, also known as the anterior and posterior surfaces of the implant, respectively. The peripheral area where the front side transitions into the back side is known as the peripheral transition area (PTA) or peripheral transition zone (PTZ). The surface area of the anterior shell can be greater than the surface area of the posterior shell, which may contribute to the projection and 3-dimensional characteristics of the device. The anterior and posterior shell thicknesses may be of similar or different thicknesses. The device can have a vertical axis or longitudinal axis, which is important for proper orientation of device.

The prosthesis may be of a round shape or an anatomical shape. The anatomical shape is also known as or described as a tear-dropped shape when viewed in profile view and may also exhibit this shape on the anterior surface in anterior to posterior view and a flat posterior surface in profile view. The round shape does not imply a sphere, but rather circular in an anterior to posterior dimension and essentially a half sphere on profile view with the posterior surface being flat or slightly contoured.

The posterior surface of the implant is the side that lies against the chest wall and the anterior surface is the side that faces towards the front of body, including the pectoralis major muscle and/or the breast tissue. The posterior surface has a roughened textured surface. The anterior surface of the implant preferably has at least two discrete textured surfaces. The superior anterior surface is relatively smooth textured and the inferior anterior surface is roughly textured. It is common when discussing texturing of implants to call the smoother textured surface the ‘smooth’ surface, and the roughened textured surface the ‘textured’ surface, even though both do have a texture. This is done to alleviate confusion. For example, the smooth texture is not as rough as the roughened texture.

### Effects of the Invention

In accordance with one exemplary aspect of the invention, the posterior shell can be roughly textured. The texturing provides an improved surface for tissue ingrowth and adherence. This textured surface induces adherence, serves to maintain the proper position and orientation of the device after implantation. Maintaining the proper position is beneficial for a more naturally appearing breast shape.

To further improve tissue ingrowth and adherence, the inferior (or lower) half of the anterior surface of the device is also roughly textured. The added texturing on the anterior side provides additional surface for tissue ingrowth and adherence. When combined with the posterior texturing, the entire back of the implant and the lower half of the implant circumferentially is essentially textured. This provides the benefit of maximal tissue adherence for the implant to the tissues under the implant and below the implant that are not in contact with the pectoralis major muscle. It serves to enhance tissue ingrowth and adherence and prevent implant position changes and reduce capsular contracture.

In accordance with another exemplary aspect of the invention, the superior aspect of the anterior shell can be smoothly textured which is of less texture or roughness or different sheen than the other more textured areas of the device. It is this smooth area of the anterior shell of the implant that comes in contact with the undersurface of the pectoralis major muscle. This smooth surface texture allows the pectoralis major muscle to glide over the implant without adhering to it and not cause any incremental migration.

In accordance with another aspect of the invention, the anterior shell surface textures vary in amounts of smooth and rough texturization. The anterior shell can be approximately divided into the upper and lower aspects, and the smooth texturing is preferably on the top or superior part of the implant and the roughly textured surface is preferably on the bottom or inferior part of the implant. In some exemplary embodiments of the invention, the location where the anterior transition area (ATA) from smooth to textured surfacing occurs can occur very abruptly or may be a gradient transition from smooth to rough. This texturing also aids in a longitudinal orientation of the device.

In accordance with another aspect of the invention, the anterior shell surface textures can vary in amounts of smooth and rough texturization. The anterior shell can be approximately divided into the upper and lower aspects, and the smooth is on the top or superior part of the implant and the roughly textured surface is on the bottom or inferior part of the implant. In some embodiments of the invention, the location (ATA) where the transition from smooth to textured surfacing occurs can occur in a horizontal orientation or an angled orientation or a curved orientation with the curve's apex located in the longitudinal axis of the implant or off to one side of the longitudinal axis. This texturing also aides in longitudinal orientation of the device.

In accordance with another exemplary aspect of the invention, the anterior shell surface textures may vary in amounts of smooth and rough texturization. The anterior shell can be approximately divided into the upper and lower aspects, and the smooth is preferably on the top or superior part of the implant and the roughly textured surface is preferably on the bottom or inferior part of the implant. In exemplary embodiments of the invention, the location (ATA) where the transition from smooth to textured surfacing occurs when viewed in profile can occur at the true equator or any point of highest projection of the implant; or at any point above or below the equator or any point above or below the highest projection in profile view. This texturing also aids in providing proper longitudinal orientation of the device.

In accordance with another exemplary aspect of the invention, the peripheral area where the front side transitions into the back side is called the peripheral transition area (PTA). The peripheral transition area of the implant will be smooth along the anterior surface existing smooth areas and textured along the anterior textured surfaces. In some exemplary embodiments of the invention, in the smooth surfaced peripheral transition areas there will be more texturing and this transition area texturing may be consistent with, or different than, the texturing that exists on the posterior surface or other areas of the device.

In some exemplary embodiments of the invention, the shell thickness may be of uniform thickness on the anterior and posterior shells. In other exemplary embodiments of the invention, the shell thicknesses may not be of uniform thickness when comparing anterior and posterior shells.

In some exemplary embodiments of the invention, the shell thickness may vary between the different surfaces of the anterior shell. The smooth superior anterior shell may be of thinner thickness than the textured aspect of the inferior anterior shell. In yet another exemplary embodiment of the invention, the shell thickness may be thicker in the smooth anterior shell than the textured inferior shell.

In yet another exemplary embodiment of the invention, the device may have three different shell thicknesses. For example, the anterior surface may have two different thicknesses and the posterior may be a different thickness than the two on the anterior surface.

Another exemplary feature of the present invention is a method for minimizing or preventing rotation or displacement of the implant in a human body. A proper implant is selected based upon anatomic measurements taken from the recipient (patient), the patient's enhancement wishes, and the implant dimensions. Then, a space is surgically created within the body that allows the relatively smooth or matte finished superior aspect of the breast implant to be placed posterior to the partially released pectoralis major muscle, and having the roughened or textured finished inferior aspect of the breast implant be placed inferior to the pectoralis major muscle and posterior to the breast gland to allow tissue adherence, thereby preventing migration or rotation. The textured posterior surface of the implant is in contact with the body and allows for tissue in growth and adherence and contributes to preventing migration or rotation of the breast implant and reduce capsular contracture. The breast implant orientation is positioned to be parallel with the longitudinal midline of the breast in an upright body position.

### BRIEF DESCRIPTION OF THE DRAWINGS

Next, the present invention will be explained in more detail in conjunction with the attached exemplary drawings, wherein:

FIG. 1 is an example of an anterior view of an anterior or frontal surface of an anatomic implant 100.

FIG. 1A is a profile or side view example of the implant 100.

FIG. 2 is an example of an anterior view of an anterior or frontal surface of a round implant 200.

FIG. 2A is a profile or side view example of the implant 200.

FIG. 3 is an example of a posterior view of an anterior or posterior or back surface of either an anatomic or round implant 300.

FIG. 4 is an example of an anterior view of an anterior or frontal surface of an implant 400 with superior apexed texturing in a lower (inferior) half of either an anatomic or round implant.

FIG. 5 is an example of an anterior view of an anterior or frontal surface of an implant 500 with horizontal texturing in a lower (inferior) half of either an anatomic or round implant.

FIGS. 6 and 6A are an anterior view of an anterior or frontal surface examples of a right (600) and left (601) specific implants, either anatomic or round implants. The texturing example depicts the oblique texturing in a lower or inferior half of the implant.

FIGS. 7 and 7A are the anterior view of an anterior or frontal surface examples of a right (700) and left (701) specific implants, either anatomic or round implants. The texturing example depicts a curved texturing in a lower or inferior half of the implants.

FIGS. 8A-8F are profile or side views examples of anatomic implants 800-805, respectively depicting different texturing positions, and angulations.

FIG. 9 is an example of an anterior view of an anterior or frontal view of a human chest and breast with implant 900 positioned below a partially divided pectoralis major muscle.

### DETAILED DESCRIPTION OF THE PREFERRED EMBODIMENTS

The present invention describes an exterior surface configuration, composed of, for example, a silicone elastomeric polymer shell that includes a roughly textured posterior surface, a peripheral zone of transition to the anterior surface, and an anterior surface that has at least two discrete textures, where a smoothest surface is that which is under the pectoralis major muscle and the roughly textured surface includes the areas that are not under the influence or contact of the pectoralis major muscle.

The two primary views utilized in the Figures are a (1) Frontal view also known as the Anterior to Posterior (A/P) view; and (2) a side view or Profile view, also known as a lateral view.

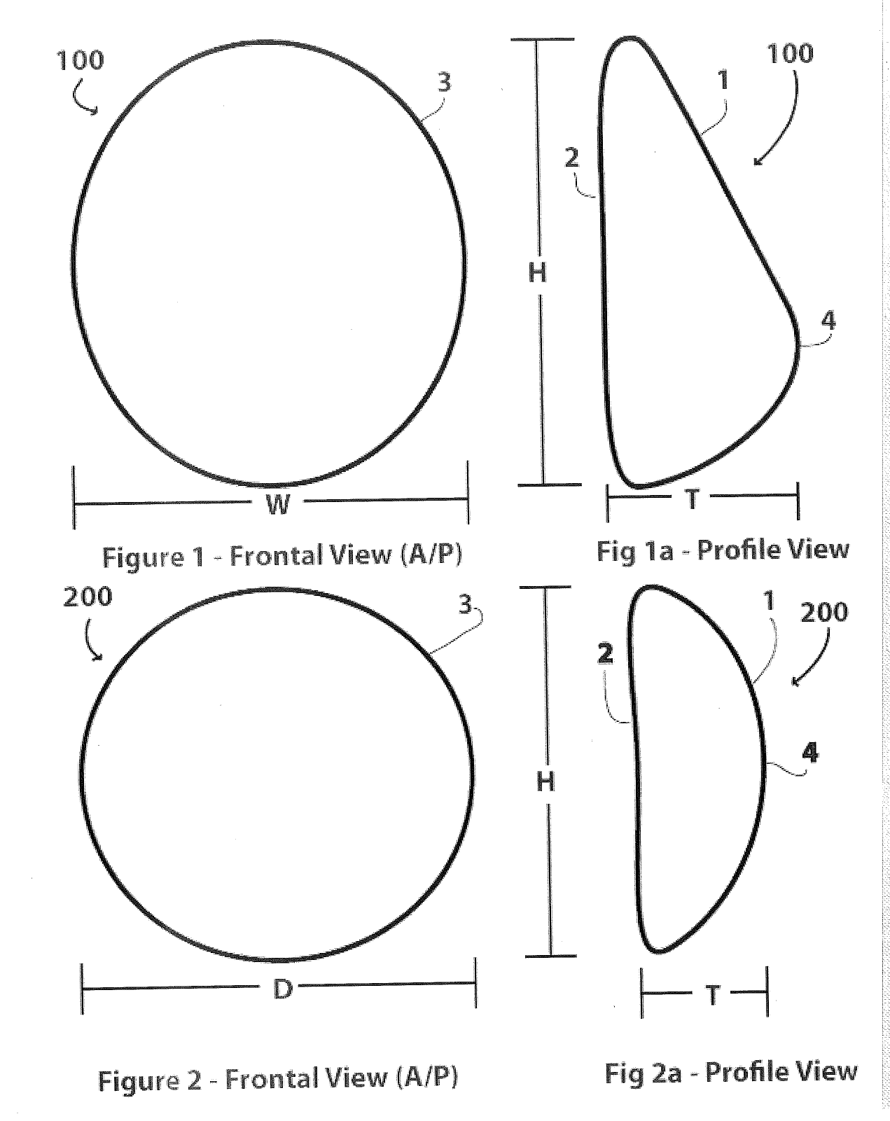

FIG. 1 depicts an example of the frontal view or anterior to posterior view (A/P) of an anatomic shaped implant 100, and FIG. 1A depicts an example of an anatomic shaped implant 100 from the side view or profile view. H represents the height of the implant. W represents the width of the device.

FIGS. 2 and 2A depict examples of the same views of a round shaped implant 200. D represents the diameter of the round implant. T represents the thickness or elevation of the implant dimension. ‘Projection’ is also a representation of the thickness, or ‘elevation’ of the implant. FIGS. 1-2A illustrate the concept of the two primary shapes of implants for breast augmentation and breast reconstruction. A breast implant can be shaped so as to be a ‘round’ or an ‘anatomic’ implant. When the adjective ‘shaped’ is used, it commonly implies an anatomic-shaped implant. Thus, the terms ‘round’ and ‘shaped’ implants may be used to describe two implant shape styles with the ‘shape’ only being designated for the anatomic implant devices.

In the figures, 1 designates the anterior surface or front exterior side of the implant; 2 designates the posterior surface or back exterior side of the implants; 3 serves to point out the peripheral transition zone (PTZ) of the implant. The front exterior side of the implant 1 includes an upper portion 22 and a lower portion 24 each having a different texture. Also, the back exterior surface of the implant 3 has a texture that is substantially smooth. That is, the back exterior surface 3 has a smoother texture than both the upper 22 and lower portion 24. The anterior or front surface of the implant shell will have a primarily smooth exterior surface on the approximately upper (superior) half of the implant shell, which comes in contact with the underside of the pectoralis major muscle.

This peripheral transition zone (PTZ) can be a discrete and essentially linear division between the front and back sides of the implant, or it can be a more gradual transition zone of greater thickness area than the linear peripheral transition zone. The PTZ only exists where there will be a transition between two textures, and thus the inferior aspect of the invention will have no transition zone because it will all be textured. Numeral 4 serves to point out a highest point of projection of the device as seen in the profile views. Note that with the anatomic implant the highest point of projection is below the midheight (median) of the implant and with the round implant the highest point of projection is at the median of the implant. With the round implant, it follows that the implant's highest point would be at the median or ‘equator’ of the device.

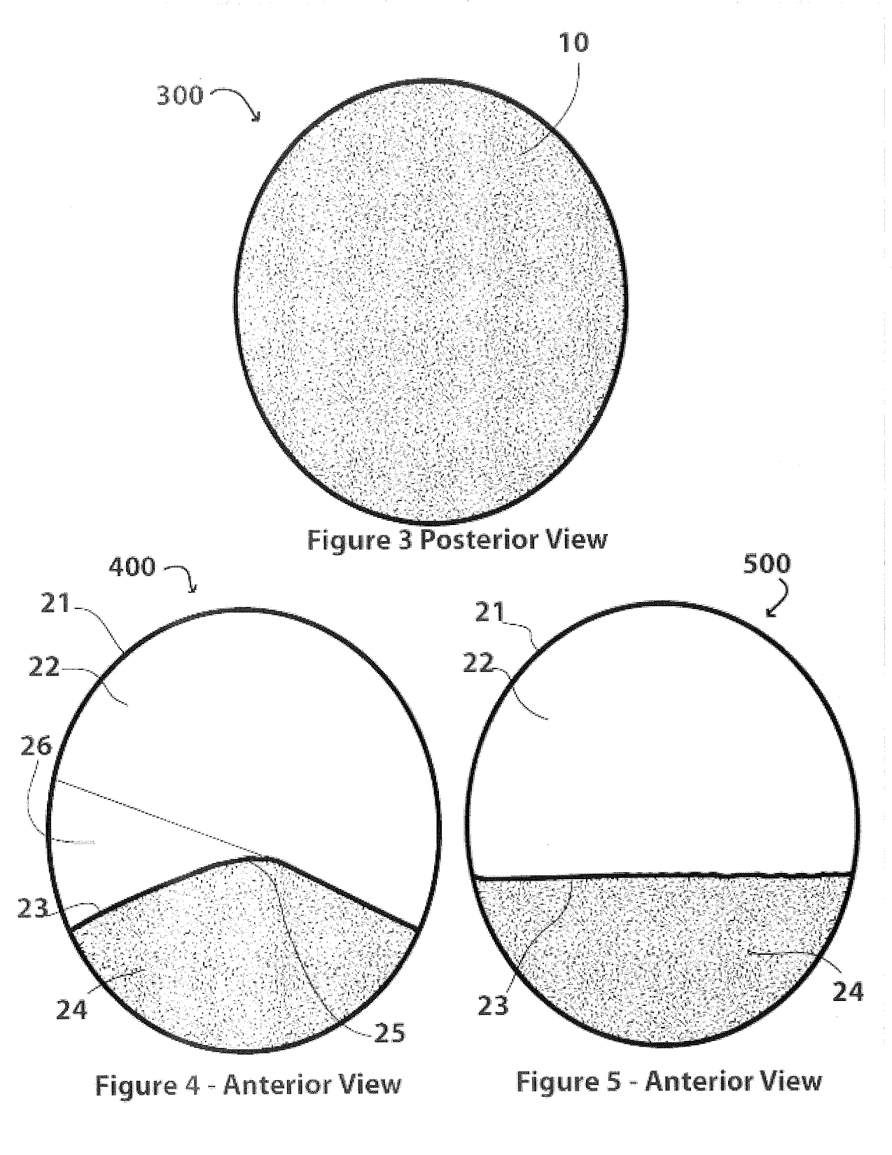

FIG. 3 depicts an example of the posterior surface view of implant 300. The texturing is exemplified graphically and identified by 10. The posterior or back of either a round or anatomic implant will have a textured exterior surface. This surface allows for tissue ingrowth and thus improved adherence to the body and prevents implant migration or malrotation.

FIG. 4 depicts an example of an anterior surface view of implant 400. This exemplary structure of the invention illustrates an implant that can be used on either the left or right breast for augmentation or reconstruction. It can be used in an anatomic-shaped implant style. It has the inferior half texturing (24) fashioned in a blunted curve or a blunted arrowhead that is apexed superiorly (25). This anterior texturing (24) is for tissue ingrowth from the surrounding tissues that are above (anterior to) the implant, e.g. the breast tissue. A part of the implant that is below (posterior to) the pectoralis major muscle and in contact with the pectoralis major muscle is smooth (22). The texture of the inferior half texturing (24) is different than the lower portion 22. There is a small area of the smooth anterior shell (26) that is not in contact with the pectoralis major muscle and yet is still smooth. This occurs so that the invention in this example is capable of being used for either breast side. This small triangular area (26) would be in contact with the breast tissue and farthest lateral from the pectoralis major muscle. In this exemplary structure the area 26 would occur if the implant would be used on the right side.

As shown in FIG. 4, an anterior transition area or “front transition area” (ATA) is designated by 23 and this zone or transition area can be an abrupt change of texturing from rough to smooth or can be a zone of gradual transition ultimately becoming smooth superiorly on the device as designated by 22.

In a similar fashion as the ATA, a peripheral transition zone (PTZ) which is designated by 21 in FIG. 4, represents the area around the periphery of the implant where there is a transition from the smooth anterior exterior surface to the textured posterior exterior surface. The anterior exterior surface will be relatively smooth or less textured compared to the roughened inferior surface area. This zone may also be a distinct, abrupt transition from smooth to textured or it may be a gradual area or zone of transition between the two exterior surface textures. In the example of implant 400, the Anterior Transition Area (ATA) is near the highest point of projection of the anatomic implant. This is the point identified in FIG. 1A on Implant 100 by reference number 4 in profile view. In other exemplary embodiments of the invention, the ATA apex may be below or above the highest point of projection of the implant.

FIG. 5 depicts an example of an anterior surface view of implant 500. This exemplary structure of the invention illustrates a round implant that can be used on either the left or right breast for augmentation or reconstruction. Although the implant is displayed with a horizontal anterior transition area (ATA) 23, the implant can be placed in the human body so that the textured anterior surface of the implant (ATA) parallels the inferior aspect of the obliquely oriented pectoralis muscle. In this instance, the implant's anterior transition line (23) would be angled and not horizontal as depicted. Since the implant is round and the base circular, it would be simply rotated into this proper position. More specifically, this particular styled implant would be turned either clockwise or counterclockwise depending on which breast it was being used for. The point being made is that the ATA 23 is a straight line relationship and not necessarily horizontal.

The anterior texturing (24) is for tissue ingrowth from the surrounding tissues that are above (anterior to) the implant, e.g. the breast tissue. The part of the implant that is below (posterior to) the pectoralis major muscle and in contact with the pectoralis major muscle is smooth (22). The anterior transition area (ATA) is designated by 23 and this zone or transition area can be an abrupt change of texturing from rough to smooth or can be a zone of gradual transition ultimately becoming smooth superiorly on the device 22.

In a similar fashion as the ATA, a peripheral transition zone (PTZ) which is designated by 21, represents the area around the periphery of the implant where there is a transition from the smooth anterior surface to the textured posterior surface. This zone may also be a distinct, abrupt transition from smooth to textured or it may be a gradual area or zone of transition between the two exterior surface textures. In the exemplary structure of implant 500, the Anterior Transition Area (ATA) is below the equator of the round implant. In other embodiments of the invention, the ATA will be below or above the equator of the implant.

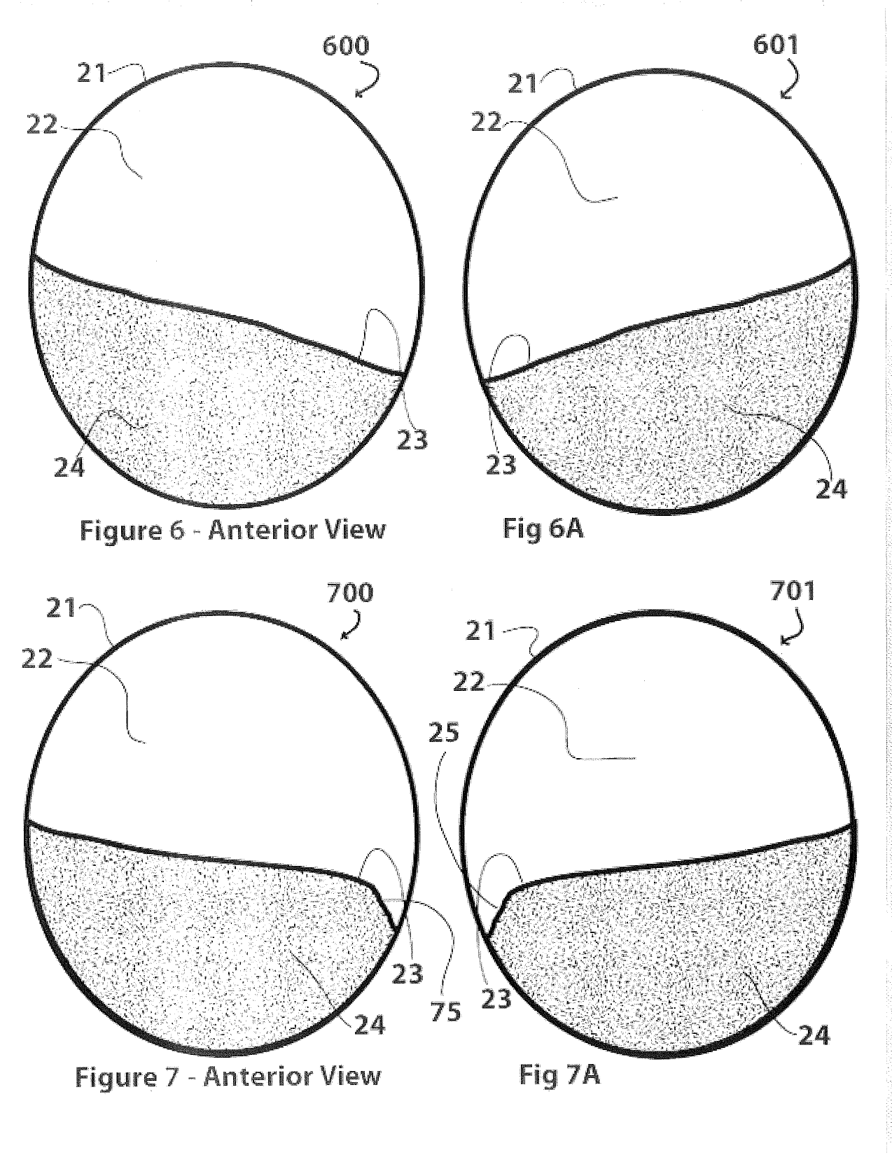

FIG. 6 represents an anterior view of an anterior or frontal surface examples of a right implant (600) and a left implant (601) side specific implants, either anatomic or round implants. The texturing example depicts the oblique texturing in a lower or inferior half of the implant. In these exemplary embodiments of the invention, the manufacturing of the devices is specific for one side of the body or the other. In the present views, these figures are all displayed as if they are being looked at on the front human body, looking at them from the front as well. Thus, implant 600 is an implant specifically designed for the right breast, and implant 601 is designed for the left breast.

In these exemplary embodiments, a lowest side of an oblique line oriented Anterior Transition Area (ATA) 23 is based toward the ‘medial’ aspect of the breast, or midline of the body or cleavage area between the breasts. The implant can be placed in the human body so that the textured anterior surface of the implant (ATA) parallels the inferior aspect of the obliquely oriented pectoralis muscle. A high side of the oblique line created by ATA would be located towards the ‘lateral’ aspect of the breast or armpit side of the breasts (i.e., farthest away from the midline). The anterior surface texturing in this example is identified by number 24. This anterior surface texturing (24) is for tissue ingrowth from the surrounding tissues that are above (anterior to) the implant, e.g. the breast tissue. The part of the implant that is below (posterior to) the pectoralis major muscle and in contact with the pectoralis major muscle is smooth (22). The anterior transition area (ATA) 23 zone or transition area can be an abrupt change of texturing from rough to smooth or can be a zone of gradual transition ultimately becoming smooth superiorly on the device 22.

In a similar fashion as the ATA, a peripheral transition zone (PTZ) 21 represents the area around the periphery of the implant where there is a transition from the smooth anterior exterior surface to the textured posterior exterior surface. This zone may also be a distinct, abrupt transition from smooth to textured, or it may be a gradual area or zone of transition between the two exterior surface textures. In the exemplary structures of implants 600 and 601, the Anterior Transition Area (ATA) is near the highest point of projection of the anatomic implant (i.e., the point identified in FIG. 1A on Implant 100 by number 4 in profile view). In other exemplary embodiments of the invention, the ATA may be below or above the highest point of projection of the implant.

FIG. 7 represents examples of an anterior view of an anterior or frontal surface of a right implant (700) and a left implant (701) side specific implants, either anatomic or round implants. The texturing example depicts an oblique texturing in a lower or inferior half of the implant with a curve. In these exemplary embodiments of the invention, the manufacturing of the devices is specific for one side of the body or the other. In the present views, these figures are all displayed as if they are being looked at on the front human body, looking at them from the front as well. Thus, implant 700 is an implant specifically designed for the right breast, and implant 701 is designed for the left breast.

In these exemplary structures, a lowest side of an oblique line oriented Anterior Transition Area (ATA) 23 is based toward the ‘medial’ aspect of the breast, or midline of the body or cleavage area between the breasts. In this exemplary embodiment of the invention, the curved portion 75 of the ATA (23) represents another option where the implant's positioning, the pectoralis major muscle relationship thereto, and the degree of pectoralis major muscle release, would dictate that the extension of the smooth surface (22) towards the surgically released muscle endpoint. The amount of surgical release of the pectoralis major muscle that is performed is based upon the patient's anatomy among other factors. Thus, this curved increase in smooth surface shows that less pectoralis muscle would be released, and therefore smoother surface is indicated to allow the overlying muscle still attached in the midline to glide over the implant without causing malrotation.

The exemplary embodiment of FIG. 7 differs from FIG. 6 where more of the pectoralis major muscle has been released so that no curvature is indicated in FIG. 6. The location of the curve and the degree of curvature would be determined by the projection, or elevation aspects of the implant's design. The implant would most likely be placed in the human body so that the textured anterior surface (ATA) of the implant parallels the inferior aspect of the obliquely oriented pectoralis muscle. A high side of the oblique line created by the ATA would be located towards the ‘lateral’ aspect of the breast or armpit side of the breasts or farthest away from the midline. The anterior surface texturing is in this example is identified by number 24 and is for tissue ingrowth from the surrounding tissues that are above (anterior to) the implant, e.g. the breast tissue. A part of the implant that is below (posterior to) the pectoralis major muscle and in contact with the pectoralis major muscle is smooth (22). The anterior transition area (ATA) 23 is a zone or transition area in which there can be an abrupt change of texturing from rough to smooth, or can be a zone of gradual transition ultimately becoming smooth superiorly on the device 22.

In a similar fashion as the ATA, the peripheral transition zone (PTZ) 21, represents the area around the periphery of the implant where there is a transition from the smooth anterior exterior surface to the textured posterior exterior surface. This zone may also be a distinct, abrupt transition from smooth to textured or it may be a gradual area or zone of transition between the two exterior surface textures. In the exemplary structures of implants 700 and 701, the Anterior Transition Area (ATA) is near the highest point of projection of the anatomic implant (i.e., the point identified in FIG. 1A on Implant 100 by reference number 4 in profile view). In other exemplary embodiments of the invention, the ATA may be below or above the highest point of projection of the implant.

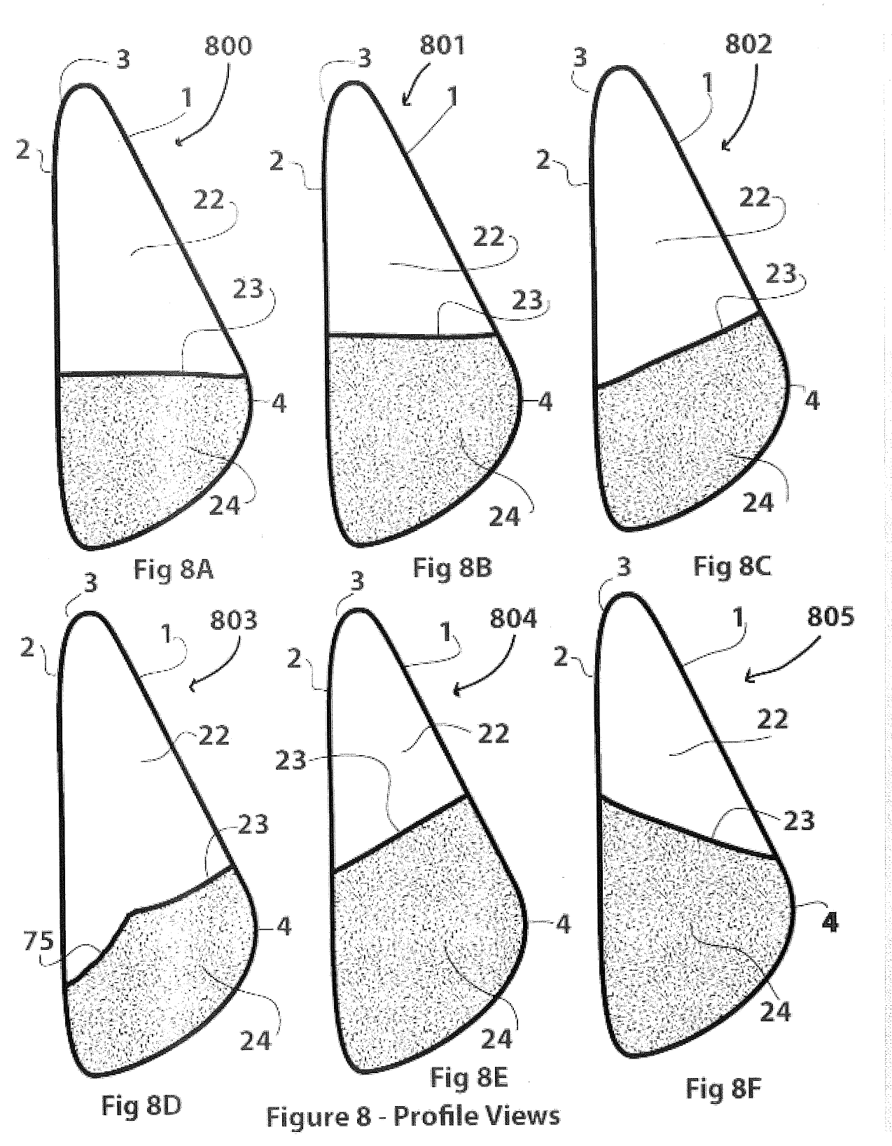

FIGS. 8A-8F depict structural examples of profile exterior views of different anatomic implants 800-805. In FIGS. 8A-8F, reference number 1 designates an anterior surface or front side of the implant; reference number 2 designates a posterior surface or back side of the implants; reference number 3 serves to point out a peripheral transition zone (PTZ) of the implant. Peripheral transition zone (PTZ) 3 can be a discrete and essentially linear division between the front and back sides of the implant, or it can be a more gradual transition zone of greater thickness area than the linear peripheral transition zone. The PTZ exists where there will be a transition between two textures, and thus the inferior (i.e., lower) aspect of the invention will have no transition zone because it will all be textured.

Reference number 4 designates the highest point of projection of the device as seen in the profile views. These examples have the inferior half texturing (24). This anterior texturing (i.e., inferior half texturing) (24) is for tissue ingrowth from the surrounding tissues that are above (anterior to) the implant, e.g. the breast tissue. A part of the implant that is below (posterior to) the pectoralis major muscle and in contact with the pectoralis major muscle is smooth (22). There is a small component of the smooth anterior shell that is not in contact with the pectoralis major muscle and yet is still smooth. This occurs so that the invention in this example is capable of being used for either breast side. This small triangular area would be in contact with the breast tissue and farthest lateral from the pectoralis major muscle. The anterior transition area (ATA) 23 is a zone or transition area which can be an abrupt change of texturing from rough to smooth, or can be a zone of gradual transition ultimately becoming smooth superiorly on the device designated by 22.

Implant 800 depicts a medial aspect of the implant. It shows the anterior transition area (ATA) 23 as being a horizontal transition. In this instance the horizontal transition would continue on to the lateral or outer aspect of the implant 800. Thus, this could also be the lateral view of implant 800 since it would look the same from either side. In this exemplary embodiment of the invention, the ATA crosses the longitudinal axis of the implant basically at or slightly above the maximum projection or elevation 4 of the implant.

Implant 801 depicts a medial aspect view of the implant. It shows the anterior transition area (ATA) 23 as being a horizontal transition. In this example, the horizontal transition may continue on to the lateral or outer aspect of the implant 801. Thus, this could also be the lateral view of implant 801 since it would look the same from either side. In this exemplary embodiment of the invention, the ATA crosses the longitudinal axis of the implant above the maximum projection or elevation 4 of the implant, although in other exemplary embodiments of the invention, it could cross the longitudinal axis of the implant at or even slightly below the maximum projection of the implant in profile view.

Implant 802 depicts a medial aspect view of the implant. It shows the anterior transition area (ATA) 23 as being an oblique transition. A lowest aspect of the oblique transition would be on the medial side of the implant 802 and towards the cleavage side or midline of the body. In this instance, the oblique transition may continue on to the lateral or outer aspect of the implant 802 and may continue to travel at an upward angle, paralleling the pectoralis major muscle. This would look similar to implant 601 in FIG. 6 in a frontal view. In the implant 802, the ATA crosses the longitudinal axis of the implant above the maximum projection or elevation 4 of the implant, although in other exemplary embodiments of the invention, it could cross the longitudinal axis of the implant at or even slightly below the maximum projection of the implant on profile view.

Implant 803 depicts a medial aspect view of the implant. It shows the anterior transition area (ATA) 23 as being an oblique transition. A lowest aspect of the oblique transition would be on the medial side of implant and towards the cleavage side or midline of the body. The anterior transition area (ATA) has a curve 75 in it medially, and this smooth area extension allows for less pectoralis muscle release and potentially better implant position medially to improve cleavage or breast definition. In this instance, the oblique transition may continue on to the lateral or outer aspect of the implant 802 and may continue to travel at an upward angle, paralleling the pectoralis major muscle. This would look similar to implant 701 in FIG. 7 in a frontal view. In the exemplary structure of implant 803, the ATA crosses the longitudinal axis of the implant above the maximum projection or elevation 4 of the implant, although in other embodiments of the invention, it could cross the longitudinal axis of the implant at or even slightly below the maximum projection of the implant on profile view.

Implant 804 depicts a medial aspect view of the implant. It shows the anterior transition area (ATA) 23 as being an oblique transition. The lowest aspect of the oblique transition would be on the medial side of the implant 804 and towards the cleavage side or midline of the body. In this instance, the oblique transition may continue on to the lateral or outer aspect of the implant 804 and may continue to travel at an upward angle, paralleling the pectoralis major muscle. This would look similar to implant 601 in FIG. 6 in frontal view. Thus, implant 804 would be an implant designed for left breast placement. In another instance of the invention, the oblique transition may continue to the midline of the longitudinal axis of the implant and then obliquely transition downward mirroring its medial side line or curve. This would create an apex or curve to the transition area at the longitudinal axis of the implant similar as seen on implant 400 in FIG. 4. This embodiment would allow for the device to be used on either breast. This may look similar to implant 400, in FIG. 4. In the exemplary structure of implant 804, the ATA crosses the longitudinal axis of the implant above the maximum projection or elevation 4 of the implant, although in other embodiments of the invention, it could cross the longitudinal axis of the implant at or even slightly below the maximum projection of the implant on profile view.

Implant 805 depicts a Lateral aspect view of the implant. It shows the anterior transition area (ATA) 23 as being an oblique transition. A lowest aspect of the oblique transition would be on the medial side of implant and towards the cleavage side or midline of the body. In this instance, the oblique transition is seen highest near the peripheral transition zone (PTZ) since this is a lateral view and not a medial view. This lateral view of implant 805 depicts an outer aspect of the implant in a profile view. The ATA may continue on to the medially towards the midline of the body. In this instance, this lateral view of the implant may represent the continuation of a few different embodiments of the invention. If the ATA continued in a straight oblique fashion, then implant 805 may resemble either implant 600 or 601 of FIG. 6, since both of these implants would look the same from their lateral views. With this same perspective, if the ATA did curve as it traveled medially, implant 805 would resemble the implants 700 or 701 of FIG. 7, but since this is a lateral view, it cannot be determined what the medial aspects of the implant 805 are without additional information. In the exemplary structure of implant 805, the ATA crosses the longitudinal axis of the implant above the maximum projection or elevation 4 of the implant although in other embodiments of the invention, it could cross the longitudinal axis of the implant at or even slightly below the maximum projection of the implant on profile view.

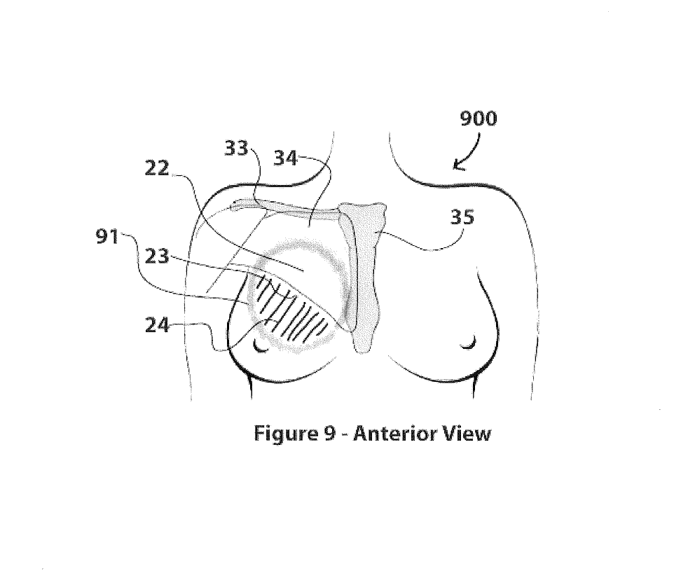

FIG. 9 depicts a frontal or anterior view of a woman's chest area with few anatomic landmarks and an exemplary implant 900 placed in the woman's chest area. The breast bone or sternum is numbered 35, and its significance is its midline position in the body and that the pectoralis major muscle (34) attaches (originates) from the sternum and ribs, and connects (inserts) on the upper arm. The clavicle 33 is a bone on the chest.

Breast implants for augmentation or reconstruction are commonly placed partially below the pectoralis major muscle. In efforts to position the implant properly, the pectoralis major muscle is partially detached from the ribs and the lowest point(s) of the sternum. The implant is then positioned under the muscle for its superior half and not under the muscle for essentially the lower half of the implant. An anterior surface of an exterior shell of the implant 900 that is under the muscle is smooth textured and is represented by reference number 22. The textured component 24 of the implant is not contacting the muscle. For additional clarification, obliquely oriented lines are drawn to symbolize the texturing of the anterior surface. The anterior transition area (ATA) is designated by reference number 23. The irregular line represented by number 91 is for contrast and represents the outermost dimensions of the implant 900.

### OTHER EMBODIMENTS

In a second exemplary embodiment, the superior aspect of the anterior shell may be smoothly textured which is of less texture or roughness or different sheen than the other more textured areas of the device. It is this smooth area of the anterior shell of the implant that comes in contact with the undersurface of the pectoralis major muscle. This smooth surface texture allows the pectoralis major muscle to glide over the implant without adhering to it and not cause any incremental migration.

In a third exemplary embodiment, the anterior shell surface textures vary in amounts of smooth and rough texturization. The anterior shell may be approximately divided into the upper and lower aspects, and the smooth texturing is preferably on the top or superior part of the implant and the roughly textured surface is preferably on the bottom or inferior part of the implant. In some exemplary embodiments of the invention, the location where the anterior transition area (ATA) from smooth to textured surfacing occurs can occur very abruptly or may be a gradient transition from smooth to rough. This texturing also aids in a longitudinal orientation of the device.

In a fourth exemplary embodiment, the anterior shell surface textures can vary in amounts of smooth and rough texturization. The anterior shell can be approximately divided into the upper and lower aspects, and the smooth is on the top or superior part of the implant and the roughly textured surface is on the bottom or inferior part of the implant. In some embodiments of the invention, the location (ATA) where the transition from smooth to textured surfacing occurs can occur in a horizontal (linear) orientation or an angled orientation or a curved orientation with the curve's apex located in the longitudinal axis of the implant or off to one side of the longitudinal axis. This texturing also aides in longitudinal orientation of the device.

In a fifth exemplary embodiment, the anterior shell surface textures may vary in amounts of smooth and rough texturization. The anterior shell can be approximately divided into the upper and lower aspects, and the smooth is preferably on the top or superior part of the implant and the roughly textured surface is preferably on the bottom or inferior part of the implant. In exemplary embodiments of the invention, the location (ATA) where the transition from smooth to textured surfacing occurs when viewed in profile can occur at the true equator or any point of highest projection of the implant; or at any point above or below the equator or any point above or below the highest projection in profile view. This texturing also aids in providing proper longitudinal orientation of the device.

In a sixth exemplary embodiment, the peripheral area where the front side transitions into the back side is called the peripheral transition zone (PTZ). The peripheral transition zone of the implant will be smooth along the anterior surface existing smooth areas and textured along the anterior textured surfaces. In some exemplary embodiments of the invention, in the smooth surfaced peripheral transition zone there will be more texturing and this transition area texturing may be consistent with, or different than, the texturing that exists on the posterior surface or other areas of the device.

In a seventh exemplary embodiment, the shell thickness may be of uniform thickness on the anterior and posterior shells. In other exemplary embodiments of the invention, the shell thicknesses may not be of uniform thickness when comparing anterior and posterior shells.

In an eighth exemplary embodiment, the shell thickness may vary between the different surfaces of the anterior shell. The smooth superior anterior shell may be of thinner thickness than the textured aspect of the inferior anterior shell. In yet another exemplary embodiment of the invention, the shell thickness may be thicker in the smooth anterior shell than the textured inferior shell.

In a ninth exemplary embodiment, the device may have three different shell thicknesses. For example, the anterior surface may have two different thicknesses and the posterior may be a different thickness than the two on the anterior surface.

Although the invention has been described with respect to the specific embodiment for complete and clear disclosure, the appended claims are not to be therefore limited but are to be construed as embodying all modifications and alternative constructions that may occur to one skilled in the art which fairly fall within the basic teaching herein set forth.

Further, Applicant's intent is to encompass the equivalents of all claim elements, and no amendment to any claim of the present application should be construed as a disclaimer of any interest in or right to an equivalent of any element or feature of the amended claim.

What is claimed is:

1. A breast prosthesis comprising:
an elastomeric shell including:
a front exterior surface including:
a first portion having a first surface texture, and
a second portion having a second surface texture different than the first surface texture, and
a back exterior surface having a third surface texture; and
a front transition area that transitions between the first portion and the second portion of the front exterior surface.

2. The breast prosthesis according to claim 1, wherein a peripheral zone is disposed at a junction of the front exterior surface and the back surface.

3. The breast prosthesis according to claim 1, wherein a surface area of the front exterior surface is greater than a surface area of the back exterior surface.

4. The breast prosthesis according to claim 1, wherein the third surface texture of the back surface and the second surface texture of the second portion of the front exterior surface is roughened, and
wherein the first surface texture of the first portion of the front exterior surface is smooth.

5. The breast prosthesis according to claim 1, wherein the back exterior surface is substantially flat.

6. The breast prosthesis according to claim 1, wherein an area of transition on the front transition area from the first surface texture to the second surface texture is either gradual or abrupt.

7. The breast prosthesis according to claim 6, wherein a location of the area of transition on the front transition area as it crosses a longitudinal axis of the elastomeric shell is at at least one of the second portion of the front exterior surface, a maximum projection of the elastomeric shell, and at any point above the maximum projection of the elastomeric shell when, viewed in a profile view.

8. The breast prosthesis according to claim 6, wherein the area of transition on the front transition area is angled with respect to a longitudinal axis of the elastomeric shell.

9. The breast prosthesis according to claim 6, wherein the area of transition on the front transition area is curved with a superior apex of the curve across the elastomeric shell and perpendicular to a longitudinal axis of the elastomeric shell.

10. The breast prosthesis according to claim 6, wherein the area of transition on the front transition area is curved with a superior apex of the curve across the elastomeric shell not apexed in a longitudinal axis of the elastomeric shell.

11. The breast prosthesis according to claim 1, wherein a shell thickness across the front exterior surface is substantially uniform.

12. The breast prosthesis according to claim 1, wherein a shell thickness across the front exterior surface is variable.

13. The breast prosthesis according to claim 1, wherein a shell thickness across the front exterior surface and a shell thickness across the back exterior surface are substantially equal.

14. The breast prosthesis according to claim 1, wherein a shell thickness across the front exterior surface is different than a shell thickness across the back exterior surface.

15. The breast prosthesis according to claim 2, wherein an area of transition on the peripheral transition zone from the front exterior surface to the back exterior surface is either gradual or abrupt.

16. The breast prosthesis according to claim 15, wherein a surface texture of the peripheral transition zone is the same as the first surface texture and the second surface texture in regions adjacent to the first surface texture and the second surface texture.

17. The breast prosthesis according to claim 1, wherein the elastomeric shell comprises an anatomic shape.

18. The breast prosthesis according to claim 17, wherein the surface area in the superior aspect of the front exterior shell is less than the area of inferior aspect of the front exterior shell.

19. The breast prosthesis according to claim 1, wherein the elastomeric shell comprises either an anatomic shape or a round shape.

20. The breast prosthesis according to claim 1, wherein the elastomeric shell comprises a round shape, and
wherein the surface of front shell is equal in superior and inferior halves of the exterior shell.

21. The breast prosthesis according to claim 19, wherein the area of transition on the front transition area is substantially straight across the front exterior surface and perpendicular to a longitudinal axis of the elastomeric shell.

22. The breast prosthesis according to claim 1, wherein the back surface texture is substantially equivalent to the front second surface texture.

23. The breast prosthesis according to claim 1, further comprising:
an additional layer having a roughened surface and being fused to the front exterior surface.

24. A method for minimizing the migration or rotation of a breast prosthesis inserted within a human body, comprising:
selecting an implant based on a measurement of a patient, a desired breast size, and a dimension of the breast prosthesis;
surgically creating a space within the patient that allows a superior aspect of the breast prosthesis to be placed posterior to a partially released pectoralis major muscle, and having a roughened or textured finished inferior aspect of the breast implant be placed inferior to a pectoralis major muscle and posterior to a breast gland to allow tissue adherence; and
placing the textured posterior surface of the implant so as to be in contact with the body and allow for tissue ingrowth and adherence and such that an orientation of the breast prosthesis is parallel with a longitudinal midline of the breast in an upright body of the patient.
